(19) United States
(12) Patent Application Publication
(10) Pub. No.: US 2024/0157103 A1
(43) Pub. Date: May 16, 2024

(54) THERAPEUTIC AGENT RELEASING MICRONEEDLE ARRAYS

(71) Applicants: Qatar Foundation for Education, Science and Community Development, Doha (QA); Qatar University, Doha (QA)

(72) Inventor: Anwarul Hasan, Doha (QA)

(21) Appl. No.: 18/389,103

(22) Filed: Nov. 13, 2023

Related U.S. Application Data
(60) Provisional application No. 63/424,704, filed on Nov. 11, 2022.

(51) Int. Cl. A61M 37/00

(52) U.S. Cl. CPC A61M 37/0015; A61M 2037/0023; A61M 2037/0061; A61M 2202/0208; A61M 2202/0275

(57) ABSTRACT

Therapeutic agent releasing microneedle arrays are described herein that comprise a hydrogel base supporting an array of microneedles formed from a hydrogel comprising a polymer and one or more therapeutic material releasing agents. The microneedle array can be formed by preparing a solution of a polymer, an oxygen releasing agent, and a nitric oxide releasing agent; applying the solution to a mold in which microneedle cavities are defined; applying a hydrogel base over the solution in the microneedle cavities, which can be of the same type of polymer as in the mold cavities however either with or without one or more therapeutic agents; centrifuging the mold with the hydrogel base; crosslinking and/or curing the microneedles array with the base; and detaching the microneedle array with the hydrogel base from the mold. This method yields an array of microneedles comprising the polymer, the oxygen releasing agent, and the nitric oxide releasing agent.

### CROSS REFERENCE TO RELATED APPLICATION

This application claims the benefit of priority to U.S. Provisional Patent Application Ser. No. 63/424,704, filed on Nov. 11, 2022, the entire disclosure of which is incorporated herein by reference in its entirety.

### FIELD

The present disclosure relates to microneedle arrays that release various therapeutic agents, such as a combination of both nitric oxide (NO) and oxygen (O₂) releasing agents.

### SUMMARY

The present disclosure provides new and innovative systems and methods of use of an array of microneedles to continuously and contemporaneously deliver therapeutic agents, such as nitric oxide (NO) and oxygen (O₂) releasing materials, to a biological subject.

In some embodiments, the microneedle arrays described herein can be used as part of a wound healing patch that addresses medical conditions, such as, but not limited to, a lack of angiogenesis (poor supply of NO), lack of cell proliferation (poor supply of oxygen) and/or microbial infection in chronic wounds to improve the treatment and healing of those wounds. The microneedle array-based wound healing patches described herein can help promote new blood vessel formation around wound sites, supply required oxygen, and aid epithelialization and fibroplasia. Moreover, the antibacterial properties of NO can also reduce the risk of microbial infection of the wound and/or wound site.

In some embodiments, microneedle array devices are described herein that comprise a hydrogel base supporting an array of microneedles formed from a hydrogel comprising a polymer and at least one therapeutic material releasing agent.

In other embodiments, a fabrication process for a microneedle array is described herein that comprises forming a solution comprising a polymer, an oxygen releasing agent, and a nitric oxide releasing agent, applying the solution to a mold in which microneedle cavities are defined; applying a hydrogel base over the solution in the microneedle cavities; centrifuging the mold and the hydrogel base; crosslinking and curing the microneedle array with the base; and detaching the cured microneedle array with the base from the mold to yield an array of microneedles including the polymer, the oxygen releasing agent, and the nitric oxide releasing agent.

The hydrogel base applied over the solution in the microneedle cavities can be of the same type of polymer as the one used in the mold. In other embodiments, the hydrogel is of a different polymer from the one used in the mold. In some embodiments, the hydrogel base can include at least one therapeutic material releasing agent. In other embodiments, the hydrogel base does not include a therapeutic material releasing agent.

In some embodiments, methods of using the microneedle array devices described herein comprise applying a microneedle array formed of a polymer, a therapeutically effective amount of an oxygen releasing agent, and a therapeutically effective amount of a nitric oxide releasing agent bonded to a hydrogel base to a wound site of a biological subject.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### DESCRIPTION OF DRAWINGS

FIGS. 1A-1B illustrate fabrication of the microneedle array patches loaded with oxygen (O₂) and nitric oxide (NO) releasing agents using two types of crosslinking techniques. FIG. 1A illustrates a GeIMA microneedle array crosslinked using visible light or UV light based crosslinking, and FIG. 1B. illustrates a Chitosan-PVA microneedle array cross-linked using chemical (TEOS) crosslinking, according to embodiments of the present disclosure.

FIG. 2 illustrates onsite action of microneedle array patches loaded with oxygen (O₂) and nitric oxide (NO) releasing agents. The microneedle array penetrates the outer layer of the skin to deliver the therapeutic agents into the deep tissue promoting cell proliferation, reepithelization and angiogenesis, according to embodiments of the present disclosure.

FIGS. 3A-3B illustrate scanning electron microscopy (SEM) images of fabricated microneedle arrays according to embodiments of the present disclosure. FIG. 3A illustrates a partial view of a 10 by 10 microneedle array patch according to embodiments of the present disclosure. FIG. 3B illustrates an image of a single microneedle from a fabricated microneedle array patch according to embodiments of the present disclosure.

### DETAILED DESCRIPTION

Described herein are microneedle array devices and methods of using the same.

In some embodiments, the devices described herein comprise a hydrogel base supporting an array of microneedles formed from a hydrogel including a polymer and at least one therapeutic material releasing agent. In some embodiments, the hydrogel includes more than one therapeutic material releasing agents.

The polymer can be a natural or synthetic polymer. Natural polymers, can include, but are not limited to, hyaluronic acid, cellulose, carrageenan, chitosan, starch, agarose, heparin, alginate, gelatin, collagen, gelatin methacryloyl (gelma), fibrin, the like, or a combination thereof. Synthetic polymers, can include, but are not limited to, poly(acrylic acid), poly(N-isopropylacrylamide), poly(ethylene glycol), poly(vinyl alcohol), poly(acrylonitrile), polyvinyl chloride, poly(HEMA) hydroxyethyl methacrylate), poly(vinylpyrrolidone), polyacrylamide, the like, or a combination thereof.

Therapeutic material releasing agents, can include, but are not limited to, an oxygen releasing agent, a nitric oxide releasing agent, a cancer treating agent, an antimicrobial agent, a painkiller, a pharmaceutical, biological material such as stem cells, induced pluripotent cells, mRNA, exosomes, extracellular vesicles, platelet rich plasma, the like, or a combination thereof.

In some embodiments, the therapeutic material releasing agents are composed of nanoparticles. In other embodiments, the therapeutic material releasing agents are composed of microparticles. In yet further embodiments, the therapeutic material releasing agents are composed of a combination of microparticles and nanoparticles or some chemically conjugated agents.

In other embodiments, microneedle array devices are described herein that comprise a hydrogel base supporting an array of microneedles formed from a solution comprising a polymer, an oxygen releasing agent, and a nitric oxide releasing agent. The oxygen releasing agent and the nitric oxide releasing agent can be comprised of nanoparticles, microparticles, or a combination thereof.

In some embodiments, microneedle array devices are described herein that comprise a hydrogel base supporting an array of microneedles formed from a solution comprising a polymer, oxygen releasing microparticles, and nitric oxide releasing nanoparticles.

In some embodiments, a microneedle array device comprises a hydrogel base supporting an array of microneedles formed from a hydrogel comprising gelatin methacryloyl (gelma), an oxygen releasing agent, and a nitric oxide releasing agent.

In other embodiments, a microneedle array device comprises a hydrogel base supporting an array of microneedles formed from a hydrogel comprising Chitosan-PVA, an oxygen releasing agent, and a nitric oxide releasing agent.

In other embodiments, the microneedle array devices described herein can deliver at least one therapeutic agent. In other embodiments, the microneedle array devices described herein can deliver more than one therapeutic agent. In some embodiments, the microneedle array devices described herein can deliver 1, 2, 3, 4, 5, at least 1, at least 2, at least 3, at least 4, at least 5, more than 1, more than 2, more than 3, more than 4, or more than 5 therapeutic agents.

The therapeutic agents can be delivered simultaneously, contemporaneously, or continuously. In other embodiments, the therapeutic material releasing agents can be delivered at intervals, e.g. within at least 1, 2, 3, 4, 5, 6, 7, 8, 9, 10 or 10 or more seconds, minutes, hours or days of each other.

Microneedle array devices described herein are capable of sustained release of therapeutic agents for enhanced and faster healing of wounds. The sustained supply of an optimized therapeutically effective amount of appropriate therapeutic agents can be used to reduce hypoxia, enhance angiogenesis, resist infection, and facilitate epithelialization and fibroplasia.

Therapeutic agents can be therapeutic material releasing agents.

In some embodiments, the microneedle array devices described herein, can be configured to carry and deliver via sustained release various medical agents to treat or prophylactically reduce the risk of various medical conditions in a biological subject. Other biomedical applications of the microneedle array devices described herein include, but are not limited to, cancer therapy, pain treatment, cardiac tissue repair (e.g., after myocardial infarction), long-term medication delivery, and the like.

In other embodiments, the microneedle array devices described herein provide the sustained and controlled delivery of NO and/or O₂ releasing material (among other wound-treating medical agents), which can be particularly useful in wound dressings to treat chronic wounds, such as, but not limited to, diabetic ulcers, burns, and other difficult-to-heal wounds. Currently, chronic wound care requires frequent and periodic change of wound dressings followed by additional application of therapeutic agents, which can be invasive to the patient. As such, described herein are also microneedle array-based patches that are minimally invasive, in that they do not lead to pain or discomfort to the patient and reduce the need for frequent replacement of dressings or visits to wound clinics, among other benefits.

Non-healing wounds are among serious complications resulting in significant morbidity and mortality, and increased health care costs, caused by prolonged microbial infections, which can compromise the patient's quality of life, increase the risk of limb amputations, and even lead to death. The delayed or absence of healing of wounds and ulcers may be the result of various factors including (i) decreased formation of blood vessel (lack of angiogenesis) due to impaired secretion of growth factors and lack of NO, (ii) poor supply of O₂ causing decreases in cell viability, proliferation and migration, and (iii) persistent microbial infections. A number of potential solutions have been investigated to solve the problem of delays in the healing of diabetic wounds including the use of bioengineered artificial skin substitutes, flap transfer, recombinant growth factors, stem cells, and combinations thereof. However, the high cost, fast degradation, rejection by the immune system and extremely slow rate of the development of blood vessels in these methods restrict their effective use. The microneedle array devices described herein provide a solution to this problem.

Microneedle array patches are described herein for pharmaceutical or therapeutic agent delivery. The present disclosure can be applied as a wound dressing material that can effectively deliver a sustained amount of a therapeutic material releasing agent. In some embodiments, the therapeutic material releasing agent can be a combination of oxygen (O₂) and nitric oxide (NO) to obtain an angiogenic effect and abundant supply of O₂, collagen deposition, epithelization, fibroplasia, and resistance to infection. The delivery of NO and O₂ using microneedle array-based patches can help promote new blood vessel formation around wound sites. Additionally, the antibacterial property of NO can also prevent or reduce the likelihood of microbial infection of the wound.

Also described herein are methods of making/the fabrication of the microneedle array-based patches described herein. In some embodiments, a method of making a microneedle array-based patch comprises forming a solution comprising a polymer, and at least one therapeutic material releasing agent; applying the solution to a mold; applying a hydrogel base over the solution in the microneedle cavities which can be comprised of the same materials as the materials inside the mold cavities or different from them; centrifuging the mold and the hydrogel base; curing the solution to the hydrogel base; and detaching the cured solution and the hydrogel base from the mold. The cured solution and the hydrogel base can be cross-linked by ultraviolet radiation or visible light based crosslinking, a form of electromagnetic radiation, the like, a combination thereof, or any other method suitable for crosslinking. In some embodiments, the microneedle arrays and the base can be cross-linked using physical, chemical or thermal crosslinking or like, or a combination there of.

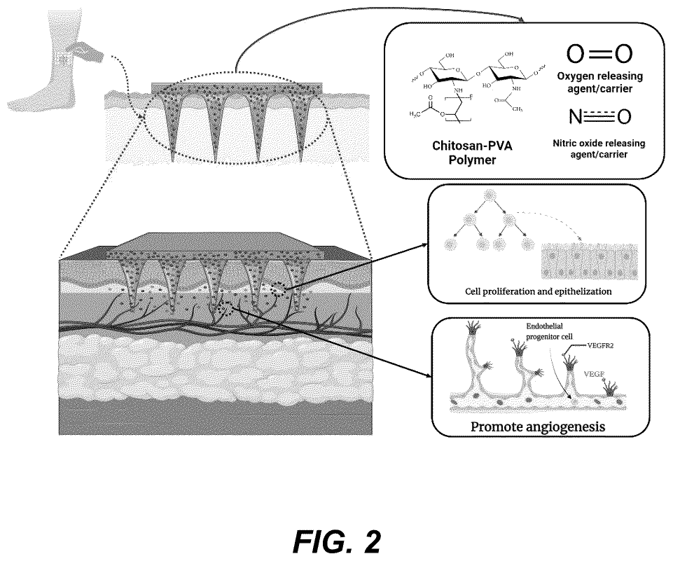

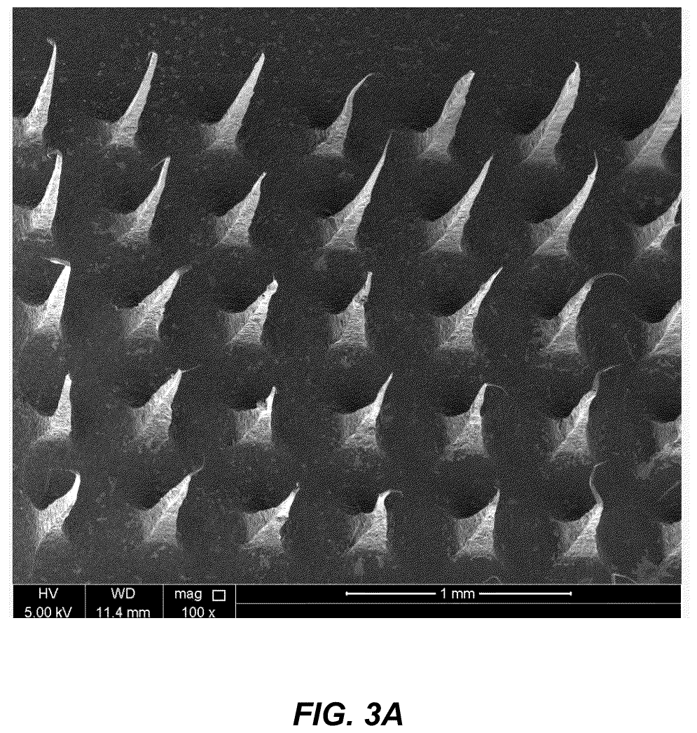

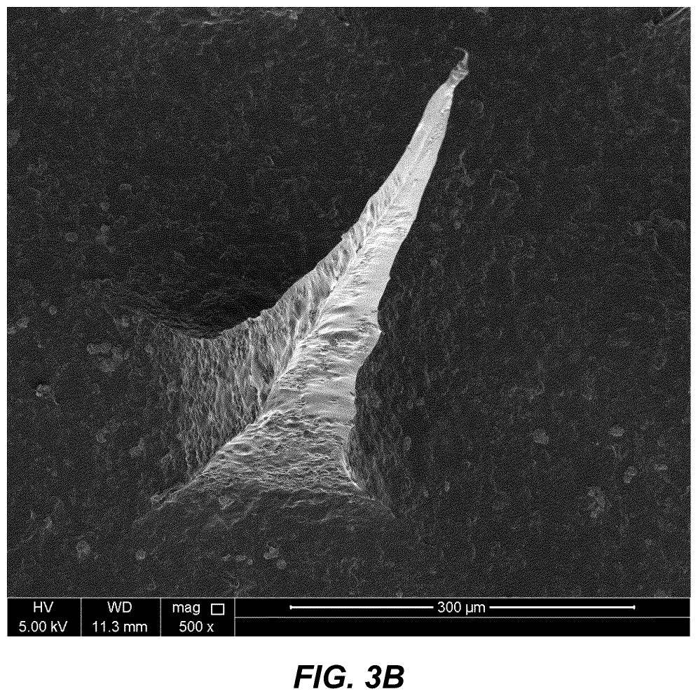

FIG. 2 illustrates onsite action of microneedle array patches loaded with oxygen (O2) and nitric oxide (NO) releasing agents. The microneedle array penetrates the outer layer of the skin to deliver the therapeutic agents into the deep tissue promoting cell proliferation, reepithelization and angiogenesis, according to embodiments of the present disclosure.

FIG. 2 is a schematic illustration presenting a visual representation of actions of oxygen and nitric oxide releasing microneedle array based patch according to embodiments of the present disclosure. In some embodiments, the patch is being applied to a diabetic wound. Upon administration, the microneedles penetrate the outer layer of the skin to deliver the therapeutics in the deep tissue as the microneedles biodegrade. The released oxygen helps in promoting cell proliferation, and eventually epithelization (the formation of a protective epithelial layer) while the released nitric oxide promotes angiogenesis (the development of new blood vessels).

The microneedle arrays described herein can act as a patch to cover the wound while the released therapeutic agents guide the healing process (overlapping the area of the wound). The therapeutic agents are incorporated in a biodegradable polymer-based hydrogel making the microneedle array, which allows the device to act as a reservoirfor local delivery of a combination of desired therapeutic agents. In some embodiments, the device co-delivers multiple therapeutic agents, including, but not limited, to nitric oxide and oxygen releasing agents.

In other embodiments, the microneedle array devices described herein can be used as part of a wound healing patch to address health issues/concerns such as, but not limited to, lack of angiogenesis (poor supply of oxygen), and microbial infection in chronic wounds to improve the treatment and healing of those wounds. The microneedle array-based patches can help promote new blood vessel formation around wound sites, supply required oxygen, and aid epithelialization and fibroplasia. Moreover, the antibacterial properties of NO also reduce the risk of microbial infection of the wound and/or wound site.

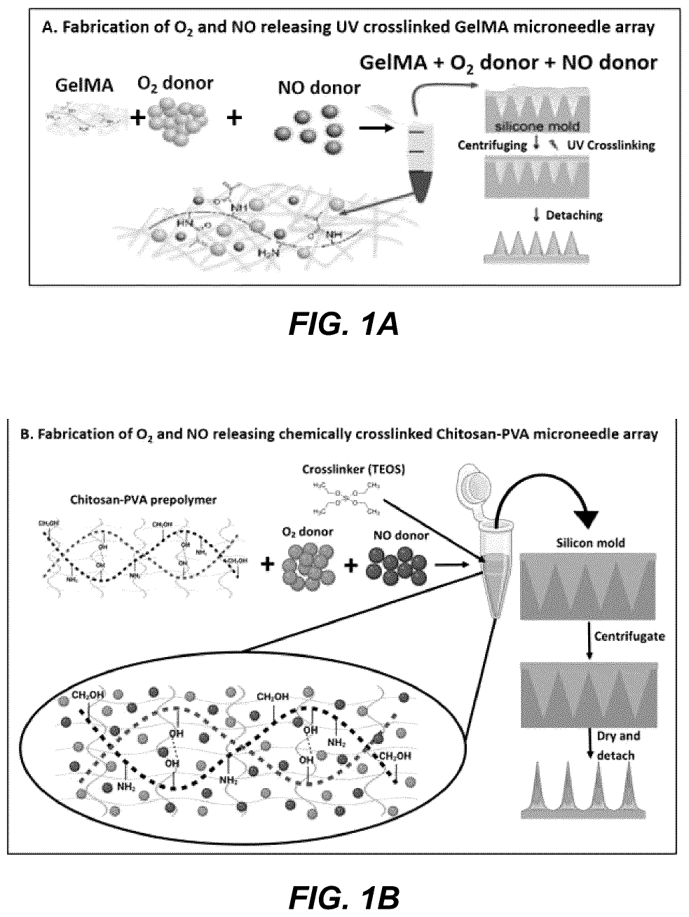

In other embodiments, a method of making a microneedle array-based patch, as illustrated in FIG. 1, comprises forming a solution comprising a polymer, an oxygen releasing agent, and a nitric oxide releasing agent; applying the solution to a mold in which microneedle cavities are defined; applying a hydrogel base over the solution in the microneedle cavities, which can be of the same type of polymer as in the mold cavities however either with or without therapeutic agent(s); centrifuging the mold and the hydrogel base; crosslinking and curing the microneedle array with the base; and detaching the cured microneedle array with the base from the mold; to yield an array of microneedles including the polymer, the oxygen releasing agent, and the nitric oxide releasing agent. The oxygen releasing agent and nitric oxide releasing agent can be comprised of nanoparticles, microparticles, or a combination thereof. In some embodiments, the oxygen releasing agent is comprised of microparticles and the nitric oxide releasing agent is comprised of nanoparticles. In some embodiments, the oxygen releasing agent is comprised of nanoparticles and the nitric oxide releasing agent is comprised of microparticles. In other embodiments, both the oxygen releasing agent and the nitric releasing agent are comprised of nanoparticles.

Described herein are new and innovative systems and methods of use of an array of microneedles to continuously and contemporaneously deliver therapeutic agents, such as nitric oxide (NO) and Oxygen (O₂) releasing materials, to a biological subject.

In some embodiments, methods of using the microneedle arrays described herein comprise applying a microneedle array formed of a polymer, a therapeutically effective amount at least one therapeutic material releasing agent bonded to a hydrogel base to a wound site of a biological subject. The at least one therapeutic releasing material agent can be, but is not limited to, an oxygen releasing agent, a nitric oxide releasing agent, a cancer treating agent, an antimicrobial agent, a painkiller, a pharmaceutical, the like, or a combination thereof.

Methods of using the microneedle arrays are described herein comprising applying a microneedle array formed of a polymer, a therapeutically effective amount of an oxygen releasing agent, and a therapeutically effect amount of a nitric oxide releasing agent bonded to a hydrogel base to a wound site of a biological subject.

Also described herein, are methods of using the microneedle arrays described herein comprise applying a microneedle array formed of a polymer, a therapeutically effective amount at least one therapeutic material releasing agent bonded to a hydrogel base to a wound site of a patient. The at least one therapeutic material releasing agent can be, but is not limited to, an oxygen releasing agent, a nitric oxide releasing agent, a cancer treating agent, an antimicrobial agent, a painkiller, a pharmaceutical, the like, or a combination thereof.

In some embodiments, method of using a microneedle array described herein comprises applying a microneedle array formed of a polymer, a therapeutically effective amount of an oxygen releasing agent, and a therapeutically effect amount of a nitric oxide releasing agent bonded to a hydrogel base to a wound site of a patient. The area of the hydrogel base can overlap an area of the wound site. In some embodiments, the hydrogel base substantially overlaps an area of the wound site. In other embodiments, the hydrogel base partially overlaps an area of the wound site.

In some embodiments, the therapeutically effective amount of the oxygen release agent can be configured based on a rate of sustained release of the oxygen releasing agent from the polymer. In other embodiments, the therapeutically effective amount of the nitric oxide release agent can be configured based on a rate of sustained release of the nitric oxide releasing agent from the polymer.

A biological subject or a patient can be a mammal. A mammal can include, but is not limited to, rats, cats, dogs, deer, monkeys, apes, bats, whales, dolphins, and humans. In some embodiments, the biological subject is a human. In other embodiments, the patient is a human.

### EXAMPLES

### Example 1: Preparation of a Microneedle Array

A solution comprising a polymer, an oxygen releasing agent, and a nitric oxide releasing agent is prepared. The solution is then applied to a mold in which microneedle cavities are defined. A hydrogel base is then applied over the solution in the microneedle cavities. The mold and hydrogel base are centrifuged together. The solution is then cured to the hydrogel base. The cured solution and the hydrogel base are then detached from the mold to yield an array of microneedles which include the polymer, the oxygen releasing agent, and the nitric oxide releasing agent.

### Example 2: Applying a Microneedle Array

A patient presents to the hospital with a wound. A medical professional attends to the patient, and applies a microneedle array, prepared from Example 1, to the wound site of the patient.

Although the present invention has been described in certain specific aspects, many additional modifications and variations would be apparent to those skilled in the art. In particular, any of the various processes described above can be performed in alternative sequences and/or in parallel (on the same or on different computing devices) in order to achieve similar results in a manner that is more appropriate to the requirements of a specific application. It is therefore to be understood that the present invention can be practiced otherwise than specifically described without departing from the scope and spirit of the present invention. Thus, embodiments of the present invention should be considered in all respects as illustrative and not restrictive. It will be evident to the annotator skilled in the art to freely combine several or all of the embodiments discussed here as deemed suitable for a specific application of the invention. Throughout this disclosure, terms like “advantageous”, “exemplary” or “preferred” indicate elements or dimensions which are particularly suitable (but not essential) to the invention or an embodiment thereof, and may be modified wherever deemed suitable by the skilled annotator, except where expressly required. Accordingly, the scope of the invention should be determined not by the embodiments illustrated, but by the appended claims and their equivalents.

What is claimed is:

1. A device, comprising:
a hydrogel base supporting an array of microneedles formed from a hydrogel including a polymer and at least one therapeutic material releasing agent.

2. The device of claim 1, wherein the at least one therapeutic material releasing agent includes a therapeutically effective amount of at least one of:
an oxygen releasing agent;
a nitric oxide releasing agent;
a cancer treating agent;
an antimicrobial agent;
a painkiller; and
a pharmaceutical.

3. The device of claim 2, wherein the at least one therapeutic material releasing agent includes a therapeutically effective amount of an oxygen releasing agent and a nitric oxide releasing agent.

4. The device of claim 1, wherein at least one of the therapeutic material releasing agent is comprised of nanoparticles.

5. The device of claim 1, wherein at least one of the therapeutic material releasing agent is comprised of microparticles.

6. The device of claim 1, wherein the at least one therapeutic material releasing agent is comprised of nanoparticles and microparticles.

7. A method, comprising:
forming a solution of a polymer, and a therapeutic material releasing agent;
applying the solution to a mold in which microneedle cavities are defined;
applying a hydrogel base over the solution in the microneedle cavities;
centrifuging the mold and the hydrogel base;
crosslinking and/or curing the microneedle array and the hydrogel base; and
detaching the cured microneedle array and the hydrogel base from the mold to yield an array of microneedles including the polymer and the therapeutic material releasing agent.

8. The method of claim 7, wherein the therapeutic material releasing agent includes an oxygen releasing agent and a nitric oxide releasing agent.

9. The method of claim 8, wherein the oxygen releasing agent and the nitric oxide releasing agent are nanoparticles.

10. The method of claim 8, wherein the oxygen releasing agent is comprised of microparticles and the nitric oxide releasing agent is comprised of nanoparticles.

11. A method, comprising:
applying a microneedle array formed of a polymer, a therapeutically effective amount of an oxygen releasing agent, and a therapeutically effective amount of a nitric oxide releasing agent bonded to a hydrogel base to a wound site of a biological subject.

12. The method of claim 11, wherein an area of the hydrogel base overlaps an area of the wound site.

13. The method of claim 11, wherein the therapeutically effective amount of the oxygen release agent is configured based on a rate of sustained release of the oxygen releasing agent from the polymer.

14. The method of claim 11, wherein the therapeutically effective amount of the nitric oxide release agent is configured based on a rate of sustained release of the nitric oxide releasing agent from the polymer.

15. The method of claim 11, wherein the biological subject is a human.

16. The method of claim 11, wherein the oxygen releasing agent and the nitric oxide releasing agent are nanoparticles.

17. The method of claim 11, wherein the oxygen releasing agent is comprised of microparticles and the nitric oxide releasing agent is comprised of nanoparticles.
